(19) United States
(12) Patent Application Publication
(10) Pub. No.: US 2005/0142087 A1
(43) Pub. Date: Jun. 30, 2005

(54) Compositions containing silicone-in-water emulsions, salts, alcohols and solvents

(76) Inventors: Yihan Liu, Midland, MI (US); Judith Mervane Vincent, Midland, MI (US)

Correspondence Address: Dow Corning Corporation;Mail Number C01232, 2200 W. Salzburg Road, P.O. Box 994, Midland, MI, 48686-0994, US

(21) Appl. No.: 10/989,847

(22) Filed: Nov. 12, 2004

Related U.S. Application Data
(63) Continuation-in-part of application No. 10/055,151, filed on Jan. 25, 2002, now abandoned.

(51) Int. Cl. A61K 7/34; A61K 7/38; A61K 7/06; A61K 7/11

(52) U.S. Cl. 424/66; 424/68; 424/70.12

(57) ABSTRACT

Silicone-in-water emulsions are prepared using a silicone polyether as the surfactant. The resulting emulsions are stable in an aqueous media in the presence of salts such as electrolytes, alcohols, solvents, and combinations thereof. The silicone-in-water emulsions are prepared by polymerizing silicon atom containing monomers in water containing the monomer, a silicone polyether, a catalyst, and optionally an organic surfactant(s), until a silicone polymer of a desired molecular weight is obtained. The silicone-in-water emulsions possess utility as additives in various coating products, personal care products, household care products, automotive care products, and petroleum products.

### CROSS-REFERENCE

This application is a continuation in part of U.S. application Ser. No. 10/055,151, filed on Jan. 25, 2002.

### FIELD OF THE INVENTION

This invention is directed to a process for preparing a silicone-in-water emulsion, the resulting silicone-in-water compositions, and to compositions further containing a salt, an alcohol, a solvent, or a combination of the salt, the alcohol, and the solvent.

### BACKGROUND OF THE INVENTION

Emulsions prepared with conventional organic surfactants are generally not stable in the presence of an alcohol or a solvent. When an ionic surfactant is used, the emulsions are not stable in the presence of salts. In fact, salts, lower alkyl alcohols, and certain organic solvents, are routinely used to break emulsions into separate phases to analyze content.

However, it has been found that when a silicone polyether is used to make a silicone-in-water emulsion, that the silicone-in-water emulsion is stable in the presence of a salt, an alcohol, an organic solvent, or a combination thereof. Such stability is an advantage and benefit in personal care, household care, automotive care, and coating industry applications.

U.S. Pat. No. 5,443,760 (Aug. 22, 1995) is directed to oil-in-water emulsions containing silicone polyethers, but the emulsions are not prepared by emulsion polymerization.

U.S. Pat. No. 5,891,954 (Apr. 6, 1999) is directed to silicone oil-in-water emulsions prepared with silicone polyethers which are stable in the presence of an alcohol, however, the silicone polyethers are post added to silicone oil-in-water emulsions prepared by emulsion polymerization. It also fails to teach the stability of such emulsions in the presence of salts and solvents.

U.S. Pat. No. 6,652,867, filed Sep. 25, 2000, entitled “Compositions Containing Organic Oil-in-Water Emulsions, Salts, Alcohols, and Solvents”, assigned to the same assignee as this invention, contains subject matter similar to subject matter disclosed herein, except that the emulsions of the '867 patent are limited to organic oils, i.e., oils containing no silicon atoms.

This invention is based on the unexpected discovery that when silicone polyethers are added during the preparation of silicone-in-water emulsions (in contrast to post addition), the resulting formulations are stable in the presence of salts such as calcium chloride and aluminum sulfate; alcohols such as methanol, ethanol, propanol and isopropanol; and organic solvents such as pentane.

### SUMMARY OF THE INVENTION

The invention provides a method of making a silicone-in-water emulsion comprising:
(i) preparing an aqueous phase containing water, a silicone polyether surfactant, and optionally one or more organic surfactants;
(ii) preparing a hydrophobic phase comprising a silicon atom containing monomer;
(iii) combining the aqueous phase and the hydrophobic phase;
(iv) adding a polymerization catalyst to the combined phases; and
(v) polymerizing the silicon atom containing monomer to a silicone polymer to form the silicone in water emulsion.

The invention also relates to the silicone-in-water emulsion compositions prepared according to the present method. The resulting silicone-in-water emulsions can be further combined with a salt, alcohol, solvent, or any combination thereof to create compositions that are stable over extended times.

### DETAILED DESCRIPTION OF THE INVENTION

The silicone-in-water emulsions of the present invention are prepared by emulsion polymerization techniques and involve mixing water, a silicone polyether, other optional surfactant(s), and silicon atom containing monomers, with a polymerization catalyst. The mixture is subjected to conditions that allow the silicon atom containing monomer to polymerize to a silicone polymer. Thus, the mixture is agitated until essentially all of the silicon atom containing monomer is polymerized, and a stable emulsion is formed. The silicone polyether is incorporated before polymerization occurs, i.e., before the polymerization catalyst is added. Processes of emulsion polymerization are described in U.S. Pat. No. 5,891,954 (Apr. 6, 1999) and U.S. Pat. No. 6,316,541 (Nov. 13, 2001), which are considered incorporated herein by reference.

Step (i) of the present invention involves preparing an aqueous phase containing water, a silicone polyether surfactant, and optionally one or more organic surfactants. Thus, the silicone polyether can be the only emulsifier used in making these emulsions, or it can be used in combination with other organic type surfactants.

Silicone Polyether (SPE) Surfactant

The silicone polyether is generally water soluble or water dispersible. It can have a rake type structure wherein the polyoxyethylene or polyoxyethylene-polyoxypropylene copolymeric units are grafted onto the siloxane backbone, or the SPE can have an ABA block copolymeric structure wherein A represents the polyether portion and B the siloxane portion of an ABA structure.

Silicone polyethers suitable for use herein have the formula M_{0-1,000}D′₁₋₁₀₀M, most preferably the formula MD₀₋₅₀₀D′₁₋₅₀M, where M represents the monofunctional unit R₃SiO_{1/2}, D represents the difunctional unit R₂SiO_{2/2}, and D′ represents the difunctional unit RR′SiO_{2/2}. In these formulas, R is an alkyl group containing 1-6 carbon atoms or an aryl group, and R′ is an oxyalkylene containing moiety. The R′ groups may contain only oxyethylene (EO) units; a combination of oxyethylene (EO) and oxypropylene (PO) units; or a combination of oxyethylene (EO) units, oxypropylene (PO) units, and oxybutylene (BO) units. Preferred R′ groups include oxyalkylene units in the approximate ratio of EO₃₋₁₀₀PO₀₋₁₀₀, most preferably in the ratio EO₃₋₃₀PO₁₋₃₀.

R′ moieties typically includes a divalent radical such as —CₘH₂ₘ— where m is 2-8 for connecting the oxyalkylene portion of R′ to the siloxane backbone. Such moieties also contain a terminating radical for the oxyalkylene portion of R′ such as hydrogen, hydroxyl, or an alkyl, aryl, alkoxy, or acetoxy group.

Silicone polyethers useful herein can also be of a type having the formula M′D_{10-1,000}D′₀₋₁₀₀M′, most preferably the formula M′D₁₀₋₅₀₀D′₀₋₅₀M′, wherein M′ represents the monofunctional unit R₂R′SiO_{1/2}, D represents the difunctional unit R₂SiO_{2/2}, and D′ represents the difunctional unit RR′SiO_{2/2}. In these formulas, R is an alkyl group containing 1-6 carbon atoms or an aryl group, and again R′ represents an oxyalkylene containing moiety. As noted previously, R′ groups typically contain only oxyethylene (EO) units or combinations of oxyethylene (EO) and oxypropylene (PO) units. Such R′ groups include these oxyalkylene units in the ratio EO₃₋₁₀₀PO₀₋₁₀₀, most preferably EO₃₋₃₀PO₁₋₃₀.

As also noted previously, R′ moieties typically include a divalent radical —CₘH₂ₘ— where m is 2-8 for connecting the oxyalkylene portions of R′ to the siloxane backbone. In addition, the moiety R′ contains a terminating radical for oxyalkylene portions of R′ such as hydrogen, hydroxyl, an alkyl, aryl, alkoxy, or acetoxy group.

In addition, silicone polyethers useful herein can be of a type having the formula MD_{0-1,000}D′₀₋₁₀₀D″_{1-1,00}M wherein D″ represents the difunctional unit RR″SiO_{2/2}, and R″ is an alkyl group containing 1-40 carbon atoms. If desired, R″ can also be an aryl group such as phenyl; an arylalkyl group such as benzyl; an alkaryl group such as tolyl; or R″ can represent a substituted alkyl group such as aminoalkyl, epoxyalkyl, or carboxyalkyl. M, D, D′, and R, are the same as defined above.

Table I shows some representative silicone polyethers according to such formulas, and these compositions are referred to in the accompanying Examples. The HLB (hydrophile-lipophile balance) of each silicone polyether is a value obtained by dividing the molecular weight percent of the ethylene oxide portion of each molecule by five.

TABLE I

| Silicone | | |
|---|---|---|
| Polyether | Nominal Structure of the Silicone Polyether | HLB |
| A | M′D₁₃M′ where R is —CH₃ and | 9.2 |
| | R′ is —(CH₂)₃(EO)₁₂OH | |
| B | MD₁₀₈D′₁₀M where R is —CH₃ and | 6.6 |
| | R′ is —(CH₂)₃(EO)₁₈(PO)₁₈OAc | |
| C | MD_{8.6}D′_{3.6}M where R is —CH₃ and | 12.3 |
| | R′ is —(CH₂)₃(EO)₁₂OH | |

The amount of silicone polyether used in the process can vary from 0.1 to 20 weight percent of the total silicone-in-water emulsion components.

Additional and/or Optional Organic Surfactant

While the silicone polyether is capable of functioning as the sole emulsifying agent, other optional and additional organic surfactants can be included in combination with the silicone polyether surfactant, if desired.

Such other surfactant can be a nonionic, cationic, anionic, amphoteric (zwitterionic), or a mixture of such surfactants. The nonionic surfactant should be a non-silicon atom containing nonionic emulsifier. Most preferred are alcohol ethoxylates R3—(OCH₂CH₂)_{c}OH, most particularly fatty alcohol ethoxylates. Fatty alcohol ethoxylates typically contain the characteristic group —(OCH₂CH₂)_{c}OH which is attached to fatty hydrocarbon residue R3 which contains about eight to about twenty carbon atoms, such as lauryl (C₁₂), cetyl (C₁₆) and stearyl (C₁₈). While the value of “c” may range from 1 to about 100, its value is typically in the range of 2 to 40.

Some examples of suitable nonionic surfactants are polyoxyethylene (4) lauryl ether, polyoxyethylene (5) lauryl ether, polyoxyethylene (23) lauryl ether, polyoxyethylene (2) cetyl ether, polyoxyethylene (10) cetyl ether, polyoxyethylene (20) cetyl ether, polyoxyethylene (2) stearyl ether, polyoxyethylene (10) stearyl ether, polyoxyethylene (20) stearyl ether, polyoxyethylene (21) stearyl ether, polyoxyethylene (100) stearyl ether, polyoxyethylene (2) oleyl ether, and polyoxyethylene (10) oleyl ether. These and other fatty alcohol ethoxylates are commercially available under names such as ALFONIC®, ARLACEL, BRIJ, GENAPOL®, LUTENSOL, NEODOL®, RENEX, SOFTANOL, SURFONIC®, TERGITOL®, TRYCOL, and VOLPO.

Cationic surfactants useful in the invention include non-silicon atom containing compounds having quaternary ammonium hydrophilic moieties in the molecule which are positively charged, such as quaternary ammonium salts represented by R4R5R6R7N⁺X^{- where R}4 to R7 are alkyl groups containing 1-30 carbon atoms, or alkyl groups derived from tallow, coconut oil, or soy; and X is halogen such as chlorine or bromine, or X can be a methosulfate group. Most preferred are (i) dialkyldimethyl ammonium salts represented by R8R9N⁺(CH₃)₂X^{-, where R}8 and R9 are alkyl groups containing 12-30 carbon atoms, or alkyl groups derived from tallow, coconut oil, or soy; and X is halogen or a methosulfate group; or (ii) monoalkyltrimethyl ammonium salts represented by R10N⁺(CH₃)₃X^{- where R}10 is an alkyl group containing 12-30 carbon atoms, or an alkyl group derived from tallow, coconut oil, or soy; and X is halogen or a methosulfate group.

Representative quaternary ammonium salts are dodecyltrimethyl ammonium bromide (DTAB), dodecyltrimethyl ammonium chloride, tetradecyltrimethyl ammonium bromide, tetradecyltrimethyl ammonium chloride, hexadecyltrimethyl ammonium bromide, hexadecyltrimethyl ammonium chloride, didodecyldimethyl ammonium bromide, dihexadecyldimethyl ammonium chloride, dihexadecyldimethyl ammonium bromide, dioctadecyldimethyl ammonium chloride, dieicosyldimethyl ammonium chloride, didocosyldimethyl ammonium chloride, dicoconutdimethyl ammonium chloride, ditallowdimethyl ammonium chloride, and ditallowdimethyl ammonium bromide. These and other quaternary ammonium salts are commercially available under names such as ADOGEN, ARQUAD, SERVAMINE, TOMAH, and VARIQUAT.

Examples of non-silicon atom containing anionic surfactants include sulfonic acids and their salt derivatives such as dodecylbenzene sulfonic acid (DBSA); alkali metal sulfosuccinates; sulfonated glyceryl esters of fatty acids such as sulfonated monoglycerides of coconut oil acids; salts of sulfonated monovalent alcohol esters such as sodium oleyl isothionate; amides of amino sulfonic acids such as the sodium salt of oleyl methyl tauride; sulfonated products of fatty acid nitriles such as palmitonitrile sulfonate; sulfonated aromatic hydrocarbons such as sodium alpha-naphthalene monosulfonate; condensation products of naphthalene sulfonic acids with formaldehyde; sodium octahydro anthracene sulfonate; alkali metal alkyl sulfates such as sodium lauryl (dodecyl) sulfate (SDS); ether sulfates having alkyl groups of eight or more carbon atoms; and alkylaryl sulfonates having one or more alkyl groups of eight or more carbon atoms.

Commercial anionic surfactants useful in this invention include triethanolamine linear alkyl sulfonate sold under the name BIO-SOFT N-300 by the Stepan Company, Northfield, Ill.; sulfates sold under the name POLYSTEP by the Stepan Company; and sodium n-hexadecyl diphenyloxide disulfonate sold under the name DOWFAX 8390 by The Dow Chemical Company, Midland, Mich.

Surfactants classified as amphoteric or zwitterionic include cocoamphocarboxy glycinate, cocoamphocarboxy propionate, cocobetaine, N-cocamidopropyldimethyl glycine, and N-lauryl-N-carboxymethyl-N-(2-hydroxyethyl)ethylene diamine. Other suitable amphoteric surfactants include the quaternary cycloimidates, betaines, and sultaines.

The betaines have the structure R11R12R13N⁺(CH₂)ₚCOO⁻ wherein R11 is an alkyl group having about twelve to eighteen carbon atoms or a mixture thereof, R12 and R13 are independently lower alkyl groups having one to three carbon atoms, and p is an integer from one to four. Specific betaines are α-(tetradecyldimethylammonio)acetate, β-(hexadecyldiethylammonio)propionate, and γ-(dodecyldimethylammonio)butyrate.

The sultaines have the structure R11R12R13N⁺(CH₂)ₚSO₃^{- wherein R}11, R12, R13, and p are as defined above. Specific useful sultaines are 3-(dodecyldimethylammonio)-propane-1-sulfonate, and 3-(tetradecyldimethylammonio)ethane-1-sulfonate.

Representative amphoteric surfactants are products sold under the names MIRATAINE® by Rhohe-Poulenc Incorporated, Cranberry, N.J.; and TEGO BETAINE by Goldschmidt Chemical Corporation, Hopewell, Va. Imidazoline and imidazoline derivatives sold under the name MIRANOL® by Rhone-Poulenc Incorporated, Cranberry, N.J. may also be employed.

The amount of optional surfactant used in the process can vary from 0.1 to 20 weight percent of the total silicone-in-water emulsion components.

Silicon Atom Containing Monomer

Step (ii) involves preparing an hydrophobic phase comprising a silicon atom containing monomer. For purposes of this invention, the silicon atom containing monomer is either a cyclic siloxane, a short chain linear siloxane, or silane capable of polymerizing to a silicone polymer of a desired molecular weight.

If a cyclic siloxane is used, it undergoes a ring opening reaction during the polymerization step, using an acid or base catalyst in the presence of water. Upon opening of the ring, siloxanes oligomers with terminal hydroxy groups are formed. These siloxane oligomers then react with one another or with other siloxane reactants that may be present in the reaction medium, through a condensation reaction, to form polysiloxane polymers or polysiloxane copolymers, i.e., silicone oils. Monomers useful in the method of this invention are those generally have limited solubility in water which can be readily polymerized using emulsion polymerization. Preferred cyclic siloxane monomers can be represented by the formula 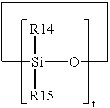
wherein R14 and R15 are each independently selected from saturated or unsaturated alkyl groups containing 1-6 carbon atoms; aryl groups containing 6-10 carbon atoms; and wherein R14 and R15 optionally can contain functional groups which are unreactive in the ring opening and polymerization reaction. Generally, t has a value of 3-7.

In particular, R14 and R15 can be represented by groups such as methyl, ethyl, propyl, phenyl, allyl, or vinyl groups; or R14 and R15 can represent groups such as —R16-F, wherein R16 is an alkylene group with 1-6 carbon atom or an arylene group with 6-10 carbon atoms, and F is a functional group such as an amine, diamine, halogen, carboxy, or mercapto group. If desired, R14 and R15 can also represent groups such as —R16-F2-R17 wherein R17 is the same as defined above for R14 and R15, and F2 is a non-carbon atom such as oxygen, nitrogen, or sulfur. Some monomers particularly preferred for this invention can be exemplified by hexamethylcyclotrisiloxane, octamethylcyclotetrasiloxane, decamethylcyclopentasiloxane, tetramethyltetravinylcyclotetrasiloxane, and tetramethyltetraphenylcyclotetrasiloxane.

It is possible to produce silicone copolymers via the emulsion polymerization reaction by having present in the reaction medium, a small portion of other types of silicon atom containing monomers. Such other monomers can be any silicon atom containing composition having hydrolyzable or silanol groups, capable of being polymerized using emulsion polymerization. Some examples of these other monomers include amine functional silanes, vinyl functional silanes, halogen alkyl functional silanes, and hydroxy endblocked polysiloxanes. In particular, they include silanol terminated polydimethysiloxanes with a degree of polymerization (DP) of 1-7; methyltrimethoxysilane; ethyltrimethoxysilane; propyltrimethoxysilane; phenyltrimethoxysilane; methylphenyldimethoxysilane; N-(2-aminoethyl)-3-aminopropyltrimethoxysilane; tetraethoxysilane; trimethoxyvinylsilane; tris-(2-methoxyethoxy)vinylsilane; and 3-chloropropryltrimethoxysilane.

If a linear siloxane oligomer is used as the silicon containing monomer, an emulsion of the linear siloxane in an aqueous phase containing the silicone polyether is first made by conventional mechanical emulsification, and the emulsion is then added with an acid or base catalyst to polymerize the linear siloxane to form polymer of the desired molecular weight. Linear siloxanes useful in the method of this invention are those generally insoluble in water which can be readily polymerized within the emulsion particle. Preferred linear siloxane monomers can be represented by the formula
HO—[R16R17SiO]ₚH
wherein R16 and R17 are each independently selected from saturated or unsaturated alkyl groups containing 1-6 carbon atoms; aryl groups containing 6-10 carbon atoms; and wherein R16 and R17 optionally can contain functional groups which are unreactive in the polymerization reaction. Generally, p has a value of 15-50.

The silicon containing monomer can also be a silane monomer selected from (i) Silane monomers of the formula Si(OR¹⁸)₄ which provide a Q unit in the silicone polymer; (ii) silane monomers of the formula R¹⁹Si(OR¹⁸)₃ which provide a T unit in the silicone polymer; (iii) silane monomers of the formula R¹⁹₂Si(OR¹⁸)₂ which provide D units; and (iv) silane monomers of the formula R¹⁹₃SiOR¹⁸ which provide M units for endblocking the silicone polymer that is formed. In the formulas, R¹⁹ can be the same or a different monovalent hydrocarbon group having 1-18 carbon atoms, or R¹⁹ can be the same or a different organofunctional substituted hydrocarbon group having 1-18 carbon atoms. R¹⁸ represents the hydrogen atom, an alkyl radical containing 1-4 carbon atoms, or one of the groups CH₃C(O)—, CH₃CH₂C(O)—, HOCH₂CH₂—, CH₃OCH₂CH₂—, or C₂H₅OCH₂CH₂—.

Some examples representative of suitable R¹⁹ groups include methyl, propyl, isobutyl, octyl, phenyl, vinyl, 3-glycidoxypropyl, aminoethylaminopropyl, 3-methacryloxypropyl, 3-chloropropyl, 3-mercaptopropyl, 3,3,3-trifluoropropyl, and perfluorobutylethyl.

If desired, a short chain trimethylsiloxy terminated polysiloxane such as hexamethyldisiloxane, or a primary alcohol such as 1-octanol, can be used in place of the silane monomer R¹⁹₃SiOR¹⁸ unit as the endblocking component.

Silicone copolymers can be produced by using more than one type of monomer, by either sequentially adding an appropriate amount of each monomer, or by the addition of a mixture of different monomers.

Short chain siloxanes, such as silane partial hydrolysis-condensation products can also be used as a starting silicon containing monomer, provided that their solubility in the aqueous medium is not unduly decreased.

Levels of the total amount of monomer useful in the emulsion polymerization process of the invention are 10-50 percent by weight, based on the combined weight of the water, the surfactant, the catalyst, and the monomer(s). The most preferred level is dependent on the nature of the monomer and the particle size being targeted.

Polymerization Catalyst

The polymerization is effected by the addition of a polymerization catalyst to the combined aqueous and hydrophobic phase. The polymerization catalyst can be a catalyst known in the art to effect siloxane polymerization. Typically though, the polymerization catalyst is a siloxane condensation catalyst. The condensation polymerization catalysts which can be used include (i) strong acids, such as substituted benzenesulfonic acids, aliphatic sulfonic acids, hydrochloric acid, and sulfuric acid; and (ii) strong bases such as quaternary ammonium hydroxides, and alkali metal hydroxides. Some ionic surfactants, such as dodecylbenzenesulfonic acid, can additionally function as a catalyst.

Typically, an acid catalyst is used to catalyze polymerization in an anionic stabilized emulsion; whereas and a basic catalyst is used to catalyze polymerization in a cationic stabilized emulsion. For nonionically stabilized emulsions, polymerization can be effected by using either an acid or basic catalyst. The amount of the catalyst present in the aqueous reaction medium should be at levels of 1×10⁻³ to 1 molarity (M). In some cases, an amine containing silane monomers such as aminoethylaminopropyltrimethoxysilane can be used as one component of the monomer mixture, and the amine functionality will catalyze the reaction without the need for an additional catalyst.

It is necessary to sufficiently mix the aqueous phase containing the silicone polyether and optional surfactant, the polymerization catalyst, and the hydrophobic phase containing the silicon monomer. The aqueous phase and hydrophobic phase can be combined completely at first, or alternatively, the hydrophobic phase can be added incrementally to the aqueous phase. If added incrementally, the exact rate of addition of the hydrophobic phase will depend on the type of monomer being used, the level of catalyst present, and the reaction temperature. The hydrophobic phase can also be mechanically emulsified in the aqueous phase by subjecting the mixture to high shear to form an emulsion before polymerization. This is necessary if the monomer is insoluble in water such that the polymerization occurs within the emulsion particle and the mechanism is termed suspension polymerization. Pre-emulsification is not necessary if monomer has a certain degree of solubility in water such that the polymerization mechanism falls in the category of emulsion polymerization.

The silicon atom containing monomer is polymerized to a silicone polymer by a polymerization reaction. Reaction times are generally less than 24 hours, and most typically less than 10 hours after combining the hydrophobic and aqueous phases. When the silicone polymer reaches the desired molecular weight, it is preferred to terminate the reaction by neutralizing the catalyst, using an equal or slightly greater stoichiometric amount of an acid or a base, for base catalyzed and acid catalyzed systems, respectively. When an amine functional silane monomer is used without the presence of another catalyst, an acid can be used to neutralize the reaction. Some appropriate acids that can be used to neutralize the reaction include strong or weak acids, such as hydrochloric acid, sulfuric acid, or acetic acid. Some appropriate bases that can be used to neutralize the reaction include strong or weak bases, such as quaternary ammonium hydroxides, alkali metal hydroxides, triethanolamine, or sodium carbonate. It is preferred to neutralize the reaction medium with a sufficient quantity of the acid or the base, such that the resulting resin containing emulsion has a pH equal to, or slightly less than 7, when a cationic surfactant is present, and a pH equal to, or slightly greater than 7, when an anionic surfactant is present.

Polymerization reaction temperatures useful according to the process of the invention are typically above the freezing point of water, but below the boiling point of water, under the operating pressure, which is normally at atmospheric pressure. Generally, the polymerization process will proceed faster at higher temperatures. The preferred temperature range is 20-95° C.

Compositions

The present invention further relates to the silicone-in-water emulsions prepared according to the methods taught herein.

The silicone-in-water emulsion contain 5-80 percent by weight of the silicone, 0.1-20 percent by weight of the surfactant(s), and the balance to 100 percent by weight being water.

Such compositions may further comprise optional components, which are added to the emulsion for various auxiliary functions.

A variety of types of silicone-in-water emulsions can be prepared according to this process. For example, microemulsions can be prepared in which the silicone oils are present as particles having a diameter of less than 140 nanometer (0.14 micrometer), preferably less than 50 nanometer (0.05 micrometer). In the case of fine emulsions, they are present as particles with diameters of 140-300 nanometer (0.14-0.30 micrometer). In standard emulsions, on the other hand, they are present as particles with diameters greater than 300 nanometer (0.30 micrometer).

Stability Measure

Emulsion stability can be evaluated by visual observation. A stable emulsion was one that did not evidence any separation or creaming effect. An unstable emulsion is indicated by the emulsion separating into an oil-rich and a water-rich layer or sedimentation.

Optional Components

Since emulsions are susceptible to microbiological contamination, a preservative may be required as an optional component of the emulsion, and some representative compounds which can be used include formaldehyde, salicylic acid, phenoxyethanol, DMDM hydantoin (1,3-dimethylol-5,5-dimethyl hydantoin), 5-bromo-5-nitro-1,3-dioxane, methyl paraben, propyl paraben, sorbic acid, imidazolidinyl urea sold under the name GERMALL® II by Sutton Laboratories, Chatham, N.J., sodium benzoate, 5-chloro-2-methyl4-isothiazolin-3-one sold under the name KATHON CG by Rohm & Haas Company, Philadelphia, Pa., and iodopropynl butyl carbamate sold under the name GLYCACIL® L by Lonza Incorporated, Fair Lawn, N.J.

A freeze/thaw stabilizer can be included as an optional component of the emulsion including compounds such as ethylene glycol, propylene glycol, glycerol, trimethylene glycol.

Another optional component is a corrosion inhibitor such as an alkanolamine, an inorganic phosphate such as zinc dithiophosphate, an inorganic phosphonate, an inorganic nitrite such as sodium nitrite, a silicate, a siliconate, an alkyl phosphate amine, a succinic anhydride such as dodecenyl succinic anhydride, an amine succinate, or an alkaline earth sulfonate such as sodium sulfonate or calcium sulfonate.

When it is desired to include an optional component in the composition, 0.01-1 percent by weight of each optional component, i.e., preservative, freeze/thaw stabilizer, or corrosion inhibitor, can be added to the composition.

The present invention further relates to compositions containing the silicone-in-water emulsions further comprising a salt component, an alcohol component, or a solvent component, in amounts as follows:
(i) 1-30 percent by weight of the salt component,
(ii) 1-80 percent by weight of the alcohol component,
(iii) 1-80 percent by weight of the solvent component, and
(iv) 10-90 percent by weight of the silicone-in-water emulsion.

Such compositions can generally be prepared at room temperature using simple propeller mixers, turbine-type mixers, Brookfield counter-rotating mixers, or homogenizing mixers. No special equipment or processing conditions are generally required.

Salt Component

As used herein, the term “salt” is intended to mean an inorganic salt or an organic salt, including compounds commonly referred to as electrolytes. Some examples of suitable inorganic salts include calcium chloride, magnesium sulfate, magnesium chloride, sodium sulfate, sodium thiosulfate, sodium chloride, sodium phosphate, ammonium chloride, ammonium carbonate, iron sulfate, aluminum sulfate, aluminum chloride, aluminum chlorohydrate, aluminum sesquichlorohydrate, aluminum dichlorohydrate, aluminum zirconium tetrachorohydrex glycine, aluminum zirconium trichlorohydrate, aluminum zirconium tetrachlorohydrate, aluminum zirconium pentachlorohydrate, and aluminum zirconium octachlorohydrate.

Some examples of suitable organic salts include sodium aluminum lactate, sodium acetate, sodium dehydroacetate, sodium butoxy ethoxy acetate, sodium caprylate, sodium citrate, sodium lactate, sodium dihydroxy glycinate, sodium gluconate, sodium glutamate, sodium hydroxymethane sulfonate, sodium oxalate, sodium phenate, sodium propionate, sodium saccharin, sodium salicylate, sodium sarcosinate, sodium toluene sulfonate, magnesium aspartate, calcium propionate, calcium saccharin, calcium d-saccharate, calcium thioglycolate, aluminum caprylate, aluminum citrate, aluminum diacetate, aluminum glycinate, aluminum lactate, aluminum methionate, aluminum phenosulfonate, potassium aspartate, potassium biphthalate, potassium bitartrate, potassium glycosulfate, potassium sorbate, potassium thioglycolate, potassium toluene sulfonate, and magnesium lactate.

Alcohol Component

The term “alcohol” as used herein is intended to mean a lower alkyl alcohol such as ethanol. Examples of some other appropriate lower alkyl alcohols which can be used are methyl alcohol, propyl alcohol, isopropyl alcohol, butyl alcohol, and isobutyl alcohol. Generally, these lower alkyl alcohols will contain one to about four carbon atoms.

Solvent Component

Solvents which can be used herein include alkanes with generally less than about 16 carbon atoms such as pentane and hexane; ketones such as acetone, methyl ethyl ketone, methyl n-butyl ketone, and methyl amyl ketone; aromatic compounds such as benzene, toluene, and ethylbenzene; esters such as ethyl acetate, isopropyl acetate, methyl acetoacetate, and isobutyl isobutyrate; ethers such as ethyl ether, butyl ethyl ether, isopentyl ether, propylene oxide, and tetrahydrofuran; glycols such as ethylene glycol, propylene glycol, and diethylene glycol; and chlorinated hydrocarbons such as methylene chloride, chloroform, carbon tetrachloride, ethyl chloride, and chlorobenzene.

### EXAMPLES

### Example I

### Mechanical Emulsification

A first portion used as Part A was prepared in a vial by adding to the vial 1.5 gram of stearic acid, 0.5 gram of glyceryl stearate and PEG-100 stearate nonionic surfactant sold under the tradename Arlacel 165, and 5 gram of decamethylcyclopentasiloxane. The contents of the vial were mixed and heated to about 80° C. to melt the surfactant. A second portion used as Part B was prepared in a 100 ml container by adding to the container 40.07 gram of deionized water, 0.93 gram of a solution containing triethanolamine as 85 percent active in water, and 2 gram of the Silicone Polyether A shown above in Table I. The contents of the container were mixed while heating to 40° C. The contents of Part A were poured slowly into Part B while continuing heating and mixing with a laboratory mixer rotating at 350 RPM. The final composition was agitated at 350 RPM for an additional 30 minutes at 40° C. An emulsion was formed and is referred to hereafter as Emulsion I.

One gram of calcium chloride salt was added to 2 gram of Emulsion I and mixed. The composition was stable. Two gram of methanol alcohol was added to 2 grams Emulsion I and shaken. The composition was stable initially but after 3 days showed partial agglomeration. Two gram of isopropanol alcohol was added to 2 gram of Emulsion I and shaken. The composition was stable initially but after 3 days showed partial agglomeration. Two gram of pentane solvent was added to 2 grams of Emulsion I and shaken. The composition separated into a clear top phase of pentane and a bottom phase of Emulsion I. The emulsion stayed intact without being extracted by the pentane phase for more than 3 days. When Silicone Polyether A was omitted from Part B in the process of making Emulsion I, it was found that the addition to Emulsion I of the same proportions of calcium chloride salt, methanol alcohol, and isopropanol alcohol, resulted in Emulsion I breaking instantly; while addition of the pentane solvent to Emulsion I extracted the silicone oil from Emulsion I.

### Example II

### Mechanical Emulsification

In a cream jar, there were combined 30 gram of deionized water, 7.5 gram of Silicone Polyether A, and 12.5 gram of a 350 centistoke (mm²/sec) polydimethylsiloxane silicone oil. The composition was sonicated with a soniprobe in a pulsed mode for one minute. An emulsion was formed and is referred to hereafter as Emulsion II. Five gram of Emulsion II was diluted with 15 ml of isopropanol alcohol and shaken. The composition was stable.

### Example III

### Mechanical Emulsification

Using the same procedure as in Example II, and by replacing Silicone Polyether A with the Silicone Polyether B shown in Table I above, another emulsion was formed, referred to hereafter as Emulsion III. 15 ml of methanol alcohol was added to 5 gram of Emulsion III and shaken. The composition was stable. The same results were obtained when ethanol alcohol or isopropanol alcohol were used in place of methanol alcohol. 15 ml of pentane solvent was added to 5 gram of Emulsion III and shaken. The composition separated into a clear top phase of pentane solvent and a bottom phase of Emulsion III. Emulsion III stayed intact without being extracted by the pentane solvent. The addition of 0.25 gram of calcium chloride salt, 12.5 ml of methanol alcohol, and 12.5 ml of pentane solvent, to 5 gram of Emulsion III, followed by shaking, produced a homogeneous emulsion showing no evidence of phase separation.

### Example IV

### Emulsion Polymerization

An oil-in-water microemulsion containing as the silicone oil, a linear hydroxy-terminated polydimethylsiloxane, was prepared by emulsion polymerization. According to the procedure, there was added to a 500 ml round bottom flask, 123.17 gram of deionized water, 28.21 gram of dodecylbenzenesulfonic acid, and 34 gram of Silicone Polyether A. The flask contents were stirred at 300 RPM while being heated at 70° C. After the surfactant had dispersed, 75 gram of octamethylcyclotetrasiloxane monomer was fed to the mixture over a 20 minutes interval and at a constant rate. The reaction was maintained at 70° C. and agitated at 300 RPM for a period of time of 5 hours measured from initiation of the monomer feed. To the mixture was then added an additional amount of 15.03 gram of Silicone Polyether A and 39 gram of deionized water. The mixture was cooled to room temperature. The reaction mixture was neutralized using 17.5 gram of triethanolamine solution with an active content of 85 percent in water. The microemulsion was preserved by the addition of 0.3 gram of Kathon CG preservative, and is referred to hereafter as Microemulsion IV. It was transparent and had a particle size of 34 nanometer. To 2 gram of Microemulsion IV was added one gram of calcium chloride salt and mixed. The composition was stable. To 2 gram of Microemulsion IV was added 2 gram of methanol alcohol and shaken. The composition became milky but remained homogenous and showed no evidence of phase separation.

### Example V

### Emulsion Polymerization

Another oil-in-water microemulsion, containing as the silicone oil a lightly crosslinked polydimethylsiloxane, was prepared by emulsion polymerization. According to the procedure, there was added to a 500 ml round bottom flask, 150.18 gram of deionized water, 28.1 gram of dodecylbenzenesulfonic acid, and 5.6 gram of the Silicone Polyether C shown above in Table I. The contents of the flask were stirred at 300 RPM while being heated to about 85° C. After the surfactant had been dispersed, 1.06 gram of crosslinking monomer tetraethoxysilane was added to the flask. There was fed to the flask, 86.97 gram of octamethylcyclotetrasiloxane monomer over an interval of 30 minutes at a constant rate. The reaction was maintained at about 85° C. and agitated at 300 RPM for another period of about 5 hours. To the flask contents was then added another 17.58 gram portion of Silicone Polyether C and an additional portion of 43.42 gram of deionized water. The flask was cooled to room temperature. To the flask was then added 18.21 gram of triethanolamine as a solution of 85 percent of the active in water, to neutralize the reaction. 0.36 gram of Kathon CG was added for preservation of the microemulsion.

The microemulsion, hereafter referred to as Microemulsion V, was translucent and had a particle size of 57 nanometer. Microemulsion V remained stable for more than 6 months. To 5 grams of Microemulsion V was added one gram of aluminum sulfate salt and mixed. The composition was stable and clear for more than 6 months. To 5 grams of Microemulsion V, was added 15 ml of methanol alcohol and shaken. The composition became milky but remained homogeneous and showed no evidence of phase separation for more than 6 months. To 5 grams of Microemulsion V was added 15 ml of ethanol alcohol and shaken. The composition became slightly milky but remained homogeneous, and showed no evidence of phase separation for more than 6 months. To 5 grams of Microemulsion V was added 15 ml of isopropanol alcohol and shaken. The composition became slightly milky but remained homogeneous, and showed no evidence of phase separation for more than 6 months. The clarity of the isopropanol alcohol diluted composition was similar in clarity obtained when water was used to dilute the microemulsion.

### Example VI

### Emulsion Polymerization to make a Silicone Resin Emulsion Incorporating a Silicone Polyether at the Start of the Process

A silicone-in-water emulsion containing as the silicone, a liquid propyl silsesquioxane, was prepared by emulsion polymerization. According to the procedure, there was added to a 500 mL round bottom flask, 232.61 gram of deionized water, 3.81 gram of dodecylbenzenesulfonic acid, and 6.0 gram of Silicone Polyether C. The flask contents were stirred at 250 RPM while being heated at 90° C. After the surfactant had dispersed, 105 gram of propyltriethoxysilane monomer was fed to the mixture over a 90 minutes interval and at a constant rate. A white emulsion gradually formed. The reaction was maintained at 90° C. and agitated at 250 RPM for a period of time of 2.5 hours measured from initiation of the monomer feed. The reaction mixture was neutralized using 3.13 gram of triethanolamine solution with an active content of 85 percent in water. The content was cooled to room temperature. The final emulsion, hereafter referred to as emulsion VI, contained approximately 19% ethanol, based on the total weight of the emulsion, which was generated as a by-product from propyltriethoxysilane hydrolysis. Emulsion VI was milky white and had an average particle size of 344 nanometer and a mono-modal particle size distribution. The emulsion remained visibly the same for more than a month at ambient condition. A sample of the emulsion was centrifuged at 2000 RPM for 30 minutes and showed no sign of separation. Emulsion VI had excellent stability upon dilution with isopropyl alcohol as evidenced by the following. To 1 gram of emulsion VI was added 4 gram of isopropyl alcohol and mixed. The composition was centrifuged at 2000 RPM for 30 minutes and showed no sign of separation; it had a transparent appearance and remained stable for days.

### Example VII

### Comparative Example of an Emulsion Polymerization to make a Silicone Resin Emulsion, NOT Incorporating a Silicone Polyether at the Start of the Process

The procedure followed in this example was the same as that of Example VI except silicone polyether C was replaced by Brij35L (laureth-23) in the same amount. A bluish white emulsion was initially observed to form during the monomer feed but turned yellowish after 80 minutes into the feed. The final emulsion, hereafter referred to as emulsion VII, contained 19% ethanol, based on the total weight of the emulsion, which was generated as a by-product from propyltriethoxysilane hydrolysis. Emulsion VII was slightly yellow and had a bi-modal particle size distribution centered about 474 nm and 3.78 μm. A significant amount of the oil phase separated out and settled on the bottom of the emulsion the next day.

### Example VIII

### Comparative Example of an Emulsion Polymerization to make a Silicone Resin Emulsion, NOT Incorporating a Silicone Polyether at the Start of the Process

The procedure used in this example was the same as used in Example VII except silicone polyether C was replaced by Brij35L and a Dean Stark trap was added to the reaction flask to simultaneously distill and remove the ethanol by-product during reaction. The final emulsion thus produced, hereafter referred to as emulsion VIII, was milky white and had an average particle size of 217 nanometers and a mono-modal particle size distribution. The emulsion remained visibly the same for more than a month at ambient condition. Emulsion VIII was immediately phase separated when diluted with methanol, ethanol or IPA at a ratio of 1 part of emulsion with 4 parts of alcohol.

Emulsions and microemulsions prepared according to this invention are useful in paper coating, textile coating, personal care, household care, automotive care, and petroleum industry, applications for delivering silicone polymers to various surfaces and substrates. For example, in personal care, they can be used in underarm products such as antiperspirants and deodorants, hair care products such as styling aids, and in products used in the care of skin.

Other variations may be made in compounds, compositions, and methods described herein without departing from the essential features of the invention. The embodiments of the invention specifically illustrated herein are exemplary only and not intended as limitations on their scope except as defined in the appended claims.

1. A method of making a silicone-in-water emulsion comprising:
(i) preparing an aqueous phase containing water, a silicone polyether surfactant, and optionally one or more organic surfactants;
(ii) preparing an hydrophobic phase comprising a silicon atom containing monomer;
(iii) combining the aqueous phase and the hydrophobic phase;
(iv) adding a polymerization catalyst to the combined phases; and
(v) polymerizing the silicon atom containing monomer to a silicone polymer to form the silicone in water emulsion.

2. A silicone-in-water emulsion prepared by the method according to claim 1.

3. A composition comprising the silicone-in-water emulsion according to claim 2 and a component selected from the group consisting of salts, alcohols, solvents, and mixtures of salts, alcohols, and solvents.

4. A composition according to claim 3 in which the component is a salt, and the salt is an inorganic salt or an organic salt selected from the group consisting of calcium chloride, magnesium sulfate, magnesium chloride, sodium sulfate, sodium thiosulfate, sodium chloride, sodium phosphate, ammonium chloride, ammonium carbonate, iron sulfate, aluminum sulfate, aluminum chloride, aluminum chlorohydrate, aluminum sesquichlorohydrate, aluminum dichlorohydrate, aluminum zirconium tetrachorohydrex glycine, aluminum zirconium trichlorohydrate, aluminum zirconium tetrachlorohydrate, aluminum zirconium pentachlorohydrate, aluminum zirconium octachlorohydrate, sodium aluminum lactate, sodium acetate, sodium dehydroacetate, sodium butoxy ethoxy acetate, sodium caprylate, sodium citrate, sodium lactate, sodium dihydroxy glycinate, sodium gluconate, sodium glutamate, sodium hydroxymethane sulfonate, sodium oxalate, sodium phenate, sodium propionate, sodium saccharin, sodium salicylate, sodium sarcosinate, sodium toluene sulfonate, magnesium aspartate, calcium propionate, calcium saccharin, calcium d-saccharate, calcium thioglycolate, aluminum caprylate, aluminum citrate, aluminum diacetate, aluminum glycinate, aluminum lactate, aluminum methionate, aluminum phenosulfonate, potassium aspartate, potassium biphthalate, potassium bitartrate, potassium glycosulfate, potassium sorbate, potassium thioglycolate, potassium toluene sulfonate, and magnesium lactate.

5. A composition according to claim 3 in which the component is an alcohol, and the alcohol component is a lower alkyl alcohol containing one to about four carbon atoms.

6. A composition according to claim 3 in which the component is a solvent, and the solvent component is an alkane containing less than about 16 carbon atoms, a ketone, an aromatic compound, an ester, an ether, a glycol, or a chlorinated hydrocarbon.

7. A product containing the silicone-in-water emulsion according to claim 2, the product being selected from the group consisting of coating products, personal care products, household care products, automotive care products, and petroleum products.

8. A method of treating the underarm, hair, or skin of the human body comprising applying to the underarm, hair, or skin of the human body, a personal care product according to claim 7.
